(19) United States
(12) Patent Application Publication
(10) Pub. No.: US 2013/0274605 A1
(43) Pub. Date: Oct. 17, 2013

(54) METHOD OF USING A DOPPLER BLOOD FLOW METER SYSTEM HAVING A FLAT PROBE APPARATUS FOR OBTAINING BLOOD FLOW DATA

(71) Applicants: Yasushi Hayashi, Kawasaki (JP); Heather Bell, Creve Coeur, MO (US)

(72) Inventors: Yasushi Hayashi, Kawasaki (JP); Heather Bell, Creve Coeur, MO (US)

(73) Assignees: Hadeco, Inc., Kawasaki (JP); Koven Technology, Inc., Creve Coeur, MO (US)

(21) Appl. No.: 13/449,133

(22) Filed: Apr. 17, 2012

(51) Int. Cl. A61B 8/06; A61B 8/04; A61B 8/02

(52) U.S. Cl. 600/454

(57) ABSTRACT

A method of obtaining blood flow data from an individual using a Doppler blood flow meter system having a main unit in operative communication with a probe apparatus having at least one flat probe configured to be attached to the individual is disclosed. The flat probe has a substantially flat configuration and includes a transmitter transducer for transmitting ultrasound signals and a receiver transducer for receiving reflected Doppler ultrasound signals. The flat probe is configured for attachment to the individual being examined. At least one inflatable cuff in operative communication with the main unit is attached to the individual and the pressure in the inflatable cuff is raised such that the blood pressure of the individual is elevated. The main unit includes a processor that processes the reflected Doppler ultrasound signals received from the flat probe to determine the systolic pressure of the individual.

### FIELD

This document relates generally to a Doppler blood flow meter system and in particular to a method of using a Doppler blood flow meter system having a flat probe apparatus for obtaining blood flow data including but not limited to systolic blood pressure.

### SUMMARY

In an embodiment, a method of obtaining blood flow data may include:
attaching a flat probe of a probe apparatus on an individual proximate or adjacent an artery;
engaging at least one inflatable pressure cuff around the individual and inflating the at least inflatable pressure cuff for elevating the blood pressure of the individual, and then deflating the at least one inflatable pressure cuff for reducing the blood pressure of the individual;
transmitting ultrasound signals through a transmitter transducer of the flat probe and receiving reflected Doppler ultrasound signals through a receiver transducer of the flat probe; and
processing the reflected Doppler ultrasound signals by a processor in operative communication with the flat probe.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a perspective view of a Doppler blood flow meter system showing the main unit and probe apparatus used in a method for obtaining blood flow data;

FIG. 2 is a side view of probe apparatus;

FIG. 3 is a bottom view of a flat probe of the probe apparatus; and

FIG. 4 is a cross-sectional view of the flat probe taken along line 4-4 of FIG. 3;

FIG. 5 is a simplified block diagram showing the various components of the Doppler blood flow meter system; and

FIG. 6 is a flow chart illustrating a method of using the Doppler blood flow meter system to obtain blood flow data from an individual.

Corresponding reference characters indicate corresponding elements among the view of the drawings. The headings used in the figures should not be interpreted to limit the scope of the claims.

### DETAILED DESCRIPTION

As described herein, a method of using a Doppler blood flow meter system to obtain blood flow data includes using a main unit operatively connected to a probe apparatus that transmits and receives ultrasound signals using a flat probe adapted for use in obtaining blood flow data from an individual. The flat probe may be positioned adjacent or proximate an artery of an individual for taking blood flow measurements which are sent to the main unit for processing and display.

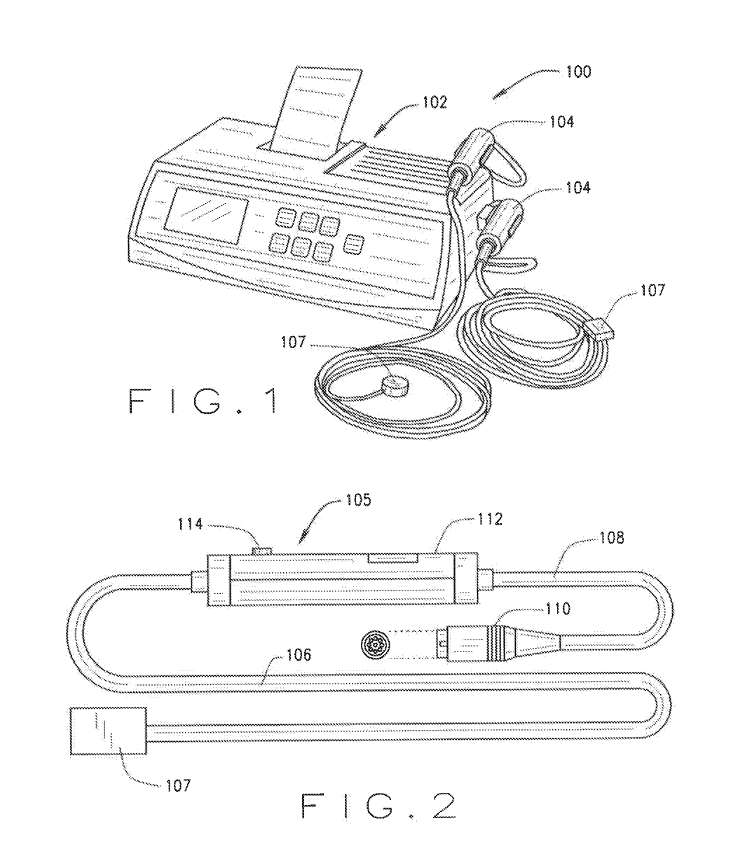

Referring to the drawings, one embodiment of a Doppler blood flow meter system for use in a method for obtaining blood flow data from an individual is illustrated and generally indicated as 100 in FIGS. 1-6. In general, the Doppler blood flow meter system 100 includes a main unit 102 operatively connected to a probe apparatus 104 through a probe cable 106 for transmitting and receiving ultrasound signals. In one embodiment, the Doppler blood flow meter system 100 may include one or more probe apparatuses 104 each having a probe housing 105 operatively connected to a flat probe 107 configured to be positioned adjacent or proximate an artery of an individual such that the blood flow through the artery may be detected by the probe apparatus 104.

As shown in FIG. 2, the probe housing 105 is operatively connected to the main unit 102 through an arrangement of a connector 110 and connector cable 108 that allows ultrasound signals to be transmitted and received by the probe apparatus 104 and processed by the main unit 102. In one embodiment, the connector 110 may be an eight-pin connector, although other types of data connectors may be used to connect the probe apparatus 104 to the main unit 102. In some embodiments, two more probe apparatuses 104 may be operatively connected to the main unit 104 for simultaneously obtaining blood flow information from more than one site along the individual.

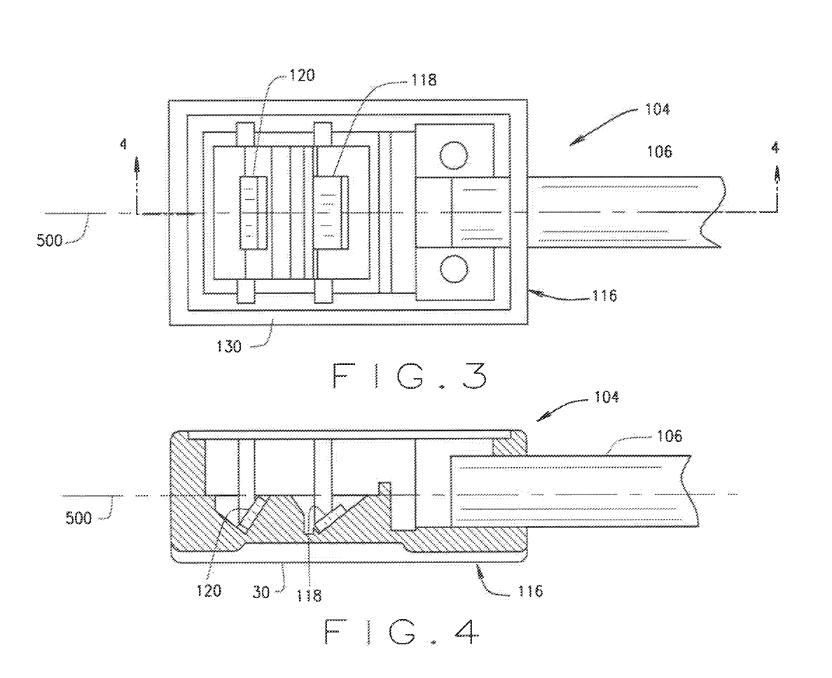

Referring to FIGS. 3 and 4, the flat probe 107 includes a casing 116 that houses a transmitter transducer 118 for transmitting ultrasound signals and a receiver transducer 120 for receiving reflected ultrasound signals. In some embodiments the bottom surface 130 of the casing 116 may form a flat or substantially flat configuration relative to longitudinal axis 500 defined by casing 116 as shown in FIG. 4. The application of the flat probe 107 to an individual eliminates the need for the flat probe 107 to be physically held after application, thereby providing hands-free operation of the probe apparatus 104 as blood flow data is being collected and processed by the main unit 102.

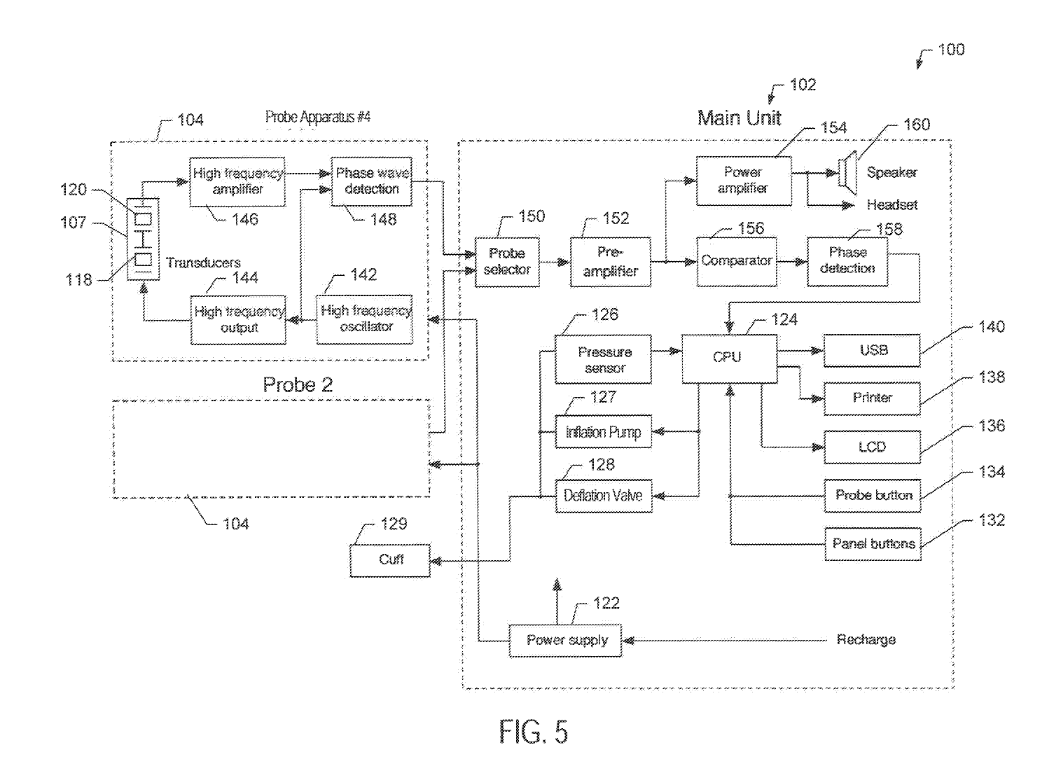

Referring to FIG. 5, one embodiment of the Doppler blood flow meter system 100 is illustrated. In this embodiment, the main unit 102 includes a power source 122 that provides power to the various components of the main unit 102. In addition, the main unit 102 includes a Central Processing Unit (CPU) 124 for controlling the various operations of the Doppler blood flow meter system 100. The CPU 124 may be in operative communication with an inflatable cuff system 125 that is used to artificially elevate an individual's blood pressure when performing one method for determining blood flow using the main unit 102 and probe apparatus 104 of the Doppler blood flow meter system 100. In one embodiment, the inflatable cuff system 125 is in operative association with an inflation pump 127 that inflates one or more inflatable blood pressure cuffs 129 wrapped around one or more limbs of an individual with air when commanded by the CPU 124. The CPU 124 also operates a deflation valve 128 in fluid flow communication with each inflatable cuff 129 for releasing air pressure inside the inflatable blood pressure cuff 129 after inflation. The main unit 102 may include a pressure sensor 126 for detecting the pressure being applied by the inflatable blood pressure cuff 129. The operative arrangement of the pump 127, relief valve 128 and pressure sensor 126 with the CPU 124 allows the cuff system 102 to perform the requisite steps for inflation and deflation of the inflatable blood pressure cuff 129 for a period of time as the blood flow data from the individual is taken through the probe apparatus 104. In some embodiments, the CPU 124 may include a probe button 134 for controlling operation of the probe apparatus.

In some embodiments, the CPU 124 is in operative communication with one or more panel buttons 132 of the main unit 102 that control the various functionalities of the Doppler blood flow meter system 100. The functionalities may include, but are not limited to, controlling an LCD display 136, a printer 138 and a USB port 140.

As further shown in FIG. 5, each probe apparatus 104 may include a high frequency oscillator 142 that produces a repetitive electronic signal that is supplied to a high frequency output component 144 which outputs a high frequency oscillation output to the transmitter transducer 118. The transmitter transducer 118 converts the high frequency oscillation output into ultrasound signals that are then transmitted from the flat probe 107 and through a biophysical object, such as an individual being examined. Once the ultrasound signals are transmitted from the transmitter transducer 118, the ultrasound signals are either transmitted through any stationary portions of the individual, such as skin and bone, or reflected by any moving portions of the individual, such as blood flow, heartbeat, etc. The reflected ultrasound signals are then received by the receiver transducer 120 of the flat probe 107 and converted from the reflected ultrasound signals to an electric Doppler shift signals which are then amplified by a high frequency amplifier 146 before being processed by a phase wave detection component 148. The phase wave detection component 148 processes the electric Doppler shift signals, which are then transmitted to the main unit 102.

Once the Doppler shifts signals are processed by the phase wave detection component 148 of the probe apparatus 104, the Doppler shift signals are then sent from the probe apparatus 104 to be processed by the main unit 102. In some embodiments, the main unit 102 may include a probe selector 150 for selecting a particular probe apparatus 104 when the main unit 102 is operatively connected to more than one probe apparatus 104 as shown in FIG. 5. The Doppler shift signals are then sent to a power amplifier 154 which further amplifies the Doppler shift signals and converts these signals into audible sounds that may be heard through either a speaker 160, or in the alternative a headset. In some embodiments, the Doppler shift signals may be processed by a filter circuit (not shown) that removes unnecessary noise and improves the signal-to-noise ratio before the Doppler shift signals are amplified and converted to audible sounds.

After the Doppler shift signals are amplified at the pre-amplifier 152, the Doppler shift signals may also be simultaneously processed by a comparator 156 and a phase wave detection component 158, which converts the Doppler shift signals to blood flow velocity wave form signals by the CPU 124. The CPU 124 may then display the blood flow velocity wave form signals at the LCD display 136 and/or printed out at printer 138.

In one method for using the inflatable cuff system 125, one or more inflatable blood pressure cuffs 129 may be wrapped around the arms, legs and/or toes of an individual in which blood flow data is to be collected. The main unit 102 is placed in either the ARMS/LEGS mode or TOE mode by actuating the appropriate panel buttons 134 on the main unit 102. The flat probe 107 is then positioned on the appropriate lower extremity artery of the individual being evaluated. Prior to inflation of the inflatable blood pressure cuffs 129, the peak amplitudes of the blood flow signals received by the CPU 124 from the probe apparatus 104 should be stable. As the pressure rises in the inflatable blood pressure cuff 129 by activation of the inflation pump 127, the blood vessel of the individual is occluded and the peak amplitudes of the blood flow signals become lower. In one embodiment, the CPU 124 determines where the peak amplitudes of the blood flow signals are below a particular threshold and then waits until the inflatable blood pressure cuffs 129 on the individual are inflated to an estimated 20 mmHg above that particular point. Once above that particular point, the CPU 124 deactivates the inflation pump 127 and allows the cuff pressure of the inflatable blood pressure cuffs 129 to lower at a moderate rate until a first signal of a return pulse that exceeds the particular threshold is detected by the probe apparatus 102. The cuff pressure at the first blood signal is considered to be the systolic pressure. After confirming a return of the rhythmical blood flow signals, the CPU 124 actuates the deflation valve 128 to release the cuff pressure and displays the systolic blood pressure on the LCD display 136.

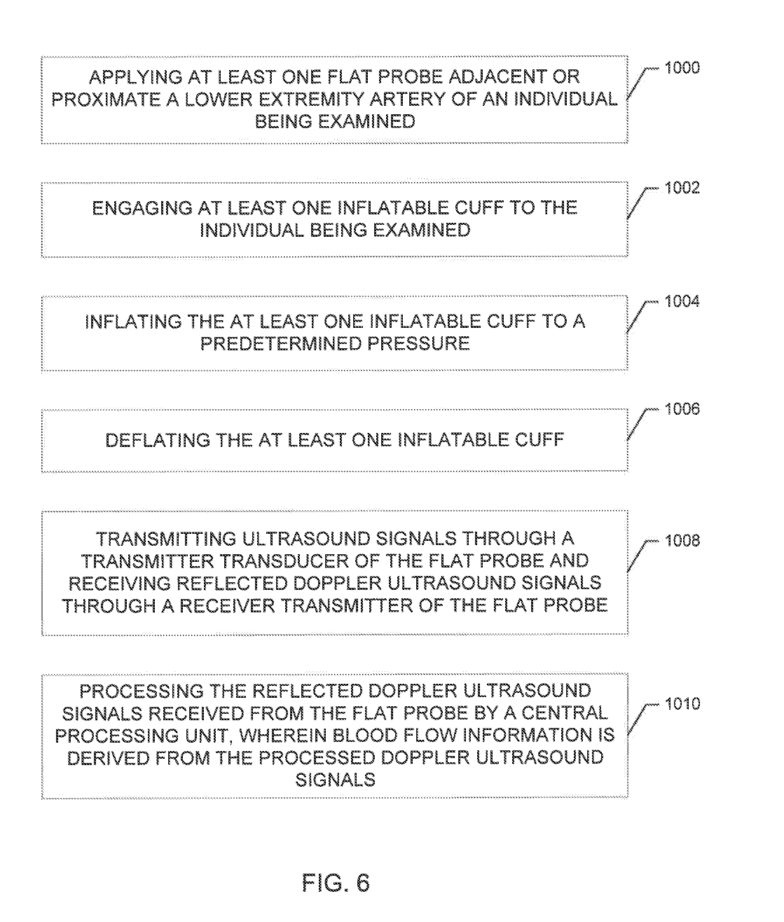

Referring to FIG. 6, a flow chart illustrates one method of using the Doppler blood flow meter system 100. In some embodiments, the method may be executed on a processor, such as CPU 124, for executing instructions for obtaining blood flow data from an individual using the Doppler blood flow meter system 100. At block 1000, at least one flat probe 107 is applied to an individual adjacent or proximate a lower extremity artery of an individual being examined. At block 1002, engaging at least one inflatable cuff 129 to the individual. At block 1004, inflating the at least one inflatable cuff 129 to a predetermined pressure, such as about 20 mmHg; At block 1006, deflating the at least one inflatable cuff 129. At block 1008, transmitting ultrasound signals through a transmitter transducer 118 of the flat probe 107 and receiving reflected Doppler ultrasound signals through a receiver transducer 120 of the flat probe 107. At block 1010, processing the reflected Doppler ultrasound signals received from the flat probe 107 by the CPU 124, wherein in one embodiment blood flow information is derived from the processed reflected Doppler ultrasound signals.

The Doppler blood flow meter system 100 may be used to detect blood flow sounds during pedal pulse checks, Ankle Brachial Index (ABI) studies, systolic pressure, segmental pressure studies, evaluation of individuals with peripheral arterial disease, and evaluation of venous valvular incompetence. In some embodiments, other types of Doppler blood flow meter systems may be utilized having a probe apparatus 104 with a flat probe 107.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of obtaining blood flow data comprising:
attaching a flat probe of a probe apparatus on an individual proximate or adjacent an artery;
engaging at least one inflatable pressure cuff around the individual and inflating the at least inflatable pressure cuff for elevating the blood pressure of the individual, and then deflating the at least one inflatable pressure cuff for reducing the blood pressure of the individual;
transmitting ultrasound signals through a transmitter transducer of the flat probe and receiving reflected Doppler ultrasound signals through a receiver transducer of the flat probe; and
processing the reflected Doppler ultrasound signals by a processor in operative communication with the flat probe.

2. The method of claim 1 further comprising determining the flow information from the reflected Doppler ultrasound signals.

3. The method of claim 2, wherein the flow information is flow rate data, flow speed data, and frequency data.

4. The method of claim 2, wherein the flow information is blood flow information from the individual.

5. The method of claim 1, further comprising monitoring the blood pressure of the individual during inflation and deflation of the at least one inflatable cuff.

6. The method of claim 1, wherein the flat probe defines a substantially flat configuration.

7. The method of claim 1, wherein the reflected Doppler ultrasound signals are reflected from a moving object.

8. The method of claim 1, wherein the moving object is blood flow or a heartbeat

9. The method of claim 1, further comprising determining a point by the processor where peak amplitudes of the reflected Doppler ultrasound signals are below a predetermined threshold.

10. The method of claim 9, further comprising allowing the pressure in the at least one inflatable pressure cuff to be inflated to about 20 mmHG above the point determined by the processor before deflating the at least one inflatable cuff such that the pressure in the at least one inflatable cuff lowers until the reflected Doppler ultrasound signal exceeds the predetermined threshold.

11. The method of claim 10, wherein a first signal of the reflected Doppler ultrasound signal that exceeds the predetermined pressure is a systolic pressure of the individual being examined.

12. The method of claim 1, further comprising converting the reflected Doppler ultrasound signals to audible signals.

13. The method of claim 1, further comprising displaying a plurality of waveforms representative of the reflected Doppler ultrasound signals.

14. The method of claim 11, wherein the processor displays the systolic pressure.
